(19) United States
(12) Patent Application Publication
(10) Pub. No.: US 2026/0108757 A1
(43) Pub. Date: Apr. 23, 2026

(54) Phototherapy Device Packaging Structure

(71) Applicant: SHENZHEN NUON MEDICAL EQUIPMENT CO., LTD., Shenzhen (CN)

(72) Inventors: ALAIN DIJKSTRA, Amstelveen (NL); LI XIANG, Shenzhen (CN); Tang Yuanzhou, Shenzhen (CN); Lin Jiaming, Shenzhen (CN)

(73) Assignee: SHENZHEN NUON MEDICAL EQUIPMENT CO., LTD., Shenzhen (CN)

(21) Appl. No.: 19/358,548

(22) Filed: Oct. 15, 2025

(30) Foreign Application Priority Data
Oct. 18, 2024 (CN) 2024225247888

(51) Int. Cl. A61N 5/06

(52) U.S. Cl. CPC A61N 5/062; A61N 2005/0632; A61N 2005/0652

(57) ABSTRACT

Embodiments of the present invention provide a phototherapy device packaging structure designed to provide an interactive user experience and reduce product trial costs. The packaging structure comprises a packaging body, a circuit board disposed within the packaging body, and a phototherapy lamp electrically connected to the circuit board and configured to emit light outward from the packaging. The packaging body may include first and second connecting portions with conductive elements, which can be brought together to form a closed circuit, thereby activating the lamp without the need for a separate switch. In certain embodiments, the packaging includes a box body and a cover with a light-transmitting portion, allowing the lamp to be used for promotional demonstration. Optional stimulation elements, such as miniature heaters, vibration modules, or aromatherapy inserts, may be integrated into the packaging to enhance user experience.

### TECHNICAL FIELD

The present invention relates generally to product packaging structures with integrated electronic components, and more particularly to a packaging body incorporating a circuit board and a light-emitting module. The invention is applicable to phototherapy devices as well as other products requiring demonstration, promotional display, or user experience functions through light emission or similar effects.

### BACKGROUND

Phototherapy devices are increasingly used in personal care, medical treatment, and cosmetic applications. With the diversification of such products, manufacturers often need to provide packaging not only for protection and storage but also for promotional and trial purposes. Conventional approaches to product trials generally require giving away actual phototherapy devices to customers, which significantly increases marketing costs and reduces overall profitability.

Moreover, traditional packaging structures merely serve as containers and lack functional interaction with the user. They do not allow customers to directly experience the light-emitting effect of the phototherapy device before purchase. As a result, users may hesitate to make purchasing decisions without first experiencing the therapeutic light output, limiting the effectiveness of promotional activities.

Therefore, there is a need for an improved packaging structure that not only protects and contains a phototherapy device but also integrates a simple light-emitting module powered by a circuit. Such a packaging structure can simulate or demonstrate the effect of a phototherapy device, allowing users to gain a preliminary experience without requiring the distribution of the actual product. This solution can reduce trial costs, enhance user engagement, and improve the efficiency of product promotion.

### OBJECTS OF THE INVENTION

Some of the objects of the invention are as follows:

An object of the present invention is to provide a phototherapy device packaging structure that reduces the cost of user experience and product promotion.

Another object of the present invention is to provide a packaging structure that integrates a circuit board and a phototherapy lamp within the packaging body, enabling light emission outward from the packaging for demonstration and trial purposes.

A further object of the present invention is to provide a packaging structure that eliminates the need to distribute actual phototherapy devices during promotional activities, thereby lowering overall marketing costs.

Still another object of the present invention is to provide a packaging structure with simple circuit and assembly features, which can be conveniently activated by structural elements such as tear lines and connecting portions without requiring complex switches.

Yet another object of the present invention is to provide a packaging structure that ensures reliable light transmission through designated light-transmitting portions of the packaging, while maintaining protective and storage functions for the phototherapy device.

### SUMMARY OF THE INVENTION

According to a first aspect of the present invention, a phototherapy device packaging structure is provided. The phototherapy device packaging structure comprising: a packaging body configured to house a phototherapy device; a circuit board disposed within the packaging body; and a phototherapy lamp mounted within the packaging body and electrically connected to the circuit board, the phototherapy lamp being configured to emit light outward from the packaging body.

In one embodiment of the invention, the phototherapy device packaging structure further comprising a battery electrically connected to the circuit board.

In one embodiment of the invention, the packaging body comprises a first connection portion and a second connection portion, each of the first and second connection portions having a conductive member electrically connected to the circuit board, and wherein the first and second connection portions are movable between a separated state and a contacting state, wherein when in the contacting state, the conductive members contact each other to form a closed electrical circuit between a power source and the phototherapy light source, thereby activating the phototherapy light source.

In one embodiment of the invention, the packaging body is a box having a first tear line and a second tear line, wherein the first connecting portion and the second connecting portion are separated from the box along the respective tear lines.

In one embodiment of the invention, the first connecting portion includes an insertion hole, and the second connecting portion includes an insertion tongue configured to engage the insertion hole when bent, thereby forming a stable mechanical and electrical connection.

In one embodiment of the invention, the conductive portions are selected from the group consisting of integrally formed metal components, metal components electrically connected via wires, and metal-plated components.

In one embodiment of the invention, the packaging body comprises a box body and a cover, the box body defining a storage cavity and a storage opening, the cover configured to close the storage opening, the cover including a light-transmitting portion, and the phototherapy lamp being aligned with the light-transmitting portion.

In one embodiment of the invention, the phototherapy device packaging structure further comprising: a protective film affixed to the cover and covering the light-transmitting portion, wherein the light-transmitting portion is exposed upon removal of the protective film.

In one embodiment of the invention, the phototherapy device packaging structure further comprising one or more stimulation elements mounted within the cover and electrically or mechanically coupled to the circuit board, the stimulation elements being selected from the group consisting of miniature heaters, vibration modules, cooling inserts, aromatherapy inserts, micro-current electrodes, EMS (Electrical Muscle Stimulation) element, PEMF (Pulsed electromagnetic field) element, and acoustic modules.

In one embodiment of the invention, the circuit board is a flexible circuit board.

In one embodiment of the invention, the phototherapy device packaging structure further comprising an electrical connector electrically connected to the circuit board and exposed outside the packaging body, the connector configured to supply external power to the phototherapy lamp.

In one embodiment of the invention, the phototherapy lamp comprises multiple LEDs configured to emit light at different wavelengths.

In one embodiment of the invention, the packaging body is selected from the group consisting of a box, pouch, strip, blister pack, or wrapping paper.

According to a second aspect of the present invention, a phototherapy system is provided. The phototherapy system comprising: a phototherapy device configured to emit therapeutic light toward a user for treatment; and a packaging structure for the phototherapy device, the packaging structure comprising: a packaging body; a circuit board disposed within the packaging body; and a phototherapy lamp mounted within the packaging body and electrically connected to the circuit board, the phototherapy lamp is configured to emit light outwardly from the packaging body.

In one embodiment of the invention, the phototherapy system further comprising a battery electrically connected to the circuit board and configured to supply electrical power to the phototherapy lamp.

In one embodiment of the invention, the phototherapy lamp of the packaging structure comprises one or more light-emitting diodes configured to emit light having wavelength characteristics substantially identical to those of the phototherapy device, whereby the emitted light replicates the treatment wavelength of the phototherapy device for demonstration or user experience.

In one embodiment of the invention, the phototherapy lamp comprises an array of light-emitting diodes spatially arranged in a pattern corresponding to an array of therapeutic emitters of the phototherapy device, whereby the emitted light field and spatial distribution pattern of the packaging structure simulate those of the phototherapy device to provide a realistic phototherapy demonstration.

In one embodiment of the invention, the packaging structure includes a first connection portion and a second connection portion, each comprising a conductive contact, the first and second connection portions being integrally formed with the packaging body and movable between a separated state and a contacting state, such that folding, bending, or pressing the packaging structure causes the conductive contacts to engage and complete a closed circuit to activate the phototherapy lamp.

In one embodiment of the invention, phototherapy system further comprising at least one skin-parameter sensor disposed within the packaging structure and configured to measure a skin parameter of the user, wherein the packaging structure is configured to transmit the measured parameter to the phototherapy device for adaptive treatment control.

According to a third aspect of the present invention, a method of operating a phototherapy system, the system comprising a phototherapy device and a packaging structure including a packaging body, a circuit board, and a phototherapy lamp mounted within the packaging body is provided. The method comprising: placing the phototherapy device within the packaging structure; activating the phototherapy lamp mounted within the packaging structure to emit light outwardly from the packaging body; emitting light from the phototherapy lamp having wavelength characteristics substantially identical to those of the phototherapy device, thereby providing a demonstration of therapeutic light emission representative of the phototherapy device; and optionally allowing a user to expose skin to the emitted light from the packaging structure to experience the therapeutic light effect without activating the phototherapy device.

In one embodiment of the invention, the method further comprising folding or pressing the packaging structure to bring a first connection portion and a second connection portion into electrical contact, thereby completing a closed circuit to power the phototherapy lamp.

In one embodiment of the invention, the method further comprising: further comprising recording user-interaction data associated with the demonstration, the data including at least one of a number of activations, duration of light emission, or user proximity, and transmitting the recorded data to a remote server or manufacturer system for analytics or marketing purposes.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “phototherapy device” refers to any device configured to emit therapeutic light for skin treatment, pain relief, or wellness applications. The device may be handheld, wearable, or a pad-type device. The invention is not limited to any particular size, shape, or light wavelength range unless explicitly stated.

In the context of the specification, the term “packaging body” refers to any structure or materials configured to house or enclose the phototherapy device for storage, shipping, or display. This includes, without limitation, boxes, pouches, strips, blister packs, wrapping paper, or cloth. The packaging body may include structural elements such as covers, cavities, openings, light-transmitting portions, connecting portions, tear lines, or slots.

In the context of the specification, the term “phototherapy lamp” refers to any light-emitting component or assembly capable of producing light energy for demonstration or mild therapeutic use. The phototherapy lamp may comprise one or more light-emitting diodes (LEDs), laser diodes, or other optoelectronic emitters. The emitted light may have wavelength characteristics substantially similar to, or representative of, those produced by the phototherapy device. In some examples, the lamp may emit visible light for display, or specific therapeutic wavelengths for functional testing.

In the context of the specification, the term “phototherapy device” denotes any therapeutic apparatus configured to emit optical radiation, such as visible light, infrared light, near-infrared light, or ultraviolet light, for treatment of biological tissue. The device may include one or more light sources, control electronics, and user interfaces for generating light with predetermined wavelength, pulse pattern, or intensity suitable for medical, cosmetic, or wellness applications.

In the context of the specification, the term “phototherapy system” refers to an integrated therapeutic assembly including at least a phototherapy device and an associated packaging structure, wherein the packaging structure may perform additional functions beyond product containment, such as light emission, user demonstration, stimulation, or sensor-based feedback.

In the context of the specification, the term “sensor” refers to any device configured to measure or detect a physical or physiological parameter, including but not limited to temperature, optical reflectance, moisture, impedance, or other skin-related properties. The sensor may communicate with the phototherapy device, a controller, or a remote server to enable adaptive control, performance logging, or analytics.

In the context of the specification, the term “circuit board” refers to any substrate configured to electrically interconnect and support components such as lamps, batteries, connectors, or stimulation elements. The circuit board may be rigid or flexible and may include conventional or printed circuitry.

In the context of the specification, the term “connection portion” refers to any structural feature of the packaging body configured to form or break an electrical circuit through physical movement or deformation. The connection portion may include conductive contacts, mechanical hinges, fold lines, or pressure-sensitive regions that, when pressed or folded, close an electrical path to activate one or more components such as the phototherapy lamp.

In the context of the specification, the term “stimulation element” refers to an electrical, photonic, vibrational, or thermal element configured to provide mild sensory feedback to the user. The stimulation element may be embedded in or coupled to the packaging body, and may be actuated concurrently with or independently of the phototherapy lamp to deliver low-level electrical pulses, temperature modulation, or light exposure to the user's skin for experiential demonstration.

In the context of the specification, the term “light-transmitting portion” refers to any region or surface of the packaging body configured to allow light emitted by the phototherapy lamp to pass through. This may include transparent, translucent, or partially transparent materials, films, or openings.

In the context of the specification, the terms “connecting portions” and “conductive portions” refer to features of the packaging body configured to form an electrical connection between components such as the battery, circuit board, and phototherapy lamp. These may include insertion tongues, sockets, metal strips, contacts, metal-plated areas, or other conductive elements.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, Super luminous diodes (“SLD”), or Organic light-emitting diodes (OLED).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several ways depending on respective wavelength ranges, all of which are envisaged to be under the scope of this invention. For example, UV radiation can be categorized as Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm). Similarly, IR radiation may also be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm).

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photobiomodulation therapy. Particular wavelengths which may be selected as the dominant emissive wavelength may include the follow, without any preference to be indicated by order: 400 nm, 405 nm, 420 nm, 430 nm, 450 nm, 465 nm, 515nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200, and 1400 nm. As used herein, the term “light therapy” refers to the use of one or more light sources of any type that emits light with a wavelength between about 400 and 1400 nm. The device may also emit blue or ultraviolet light for surface-level treatments such as acne reduction or microbial control.

The red light (approximately 630-660 nm) penetrates deeply into the scalp to stimulate blood circulation and enhance hair follicle activity, thus promoting hair growth and repair. Blue light (around 415-470 nm) exhibits antibacterial properties and is effective in treating scalp acne and reducing inflammation. Green light (approximately 520-540 nm) can help reduce pigmentation and soothe sensitive or irritated scalp tissue. Yellow light (around 580-600 nm) improves oxygen exchange in the cells and aids in detoxifying the scalp, while near-infrared light (800-850 nm) reaches deeper layers to accelerate healing and reduce pain.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 illustrates a schematic structural view of a phototherapy device packaging structure, showing the packaging body, circuit board, and phototherapy lamp arrangement, in accordance with an embodiment of the present invention.

FIG. 2 illustrates a cross-sectional view of the cover portion of FIG. 1, showing the relative positioning of the circuit board, phototherapy lamp, and the light-transmitting portion, in accordance with an embodiment of the present invention.

FIG. 3 illustrates a schematic view of the cover of FIG. 1 after removal of the protective film, showing the exposed light-transmitting portion for user experience, in accordance with an embodiment of the present invention.

FIG. 4 illustrates a schematic view of the opposite side of the cover shown in FIG. 3, showing the arrangement of a first connecting portion and a second connecting portion, in accordance with an embodiment of the present invention.

FIG. 5 illustrates a schematic view of the cover of FIG. 4 in a bent state, showing the contact between the first and second conductive portions to form a closed circuit, in accordance with an embodiment of the present invention.

FIG. 6 illustrates a schematic view of the circuit board, the first conductive portion, and the second conductive portion in the bent state of FIG. 5, showing the electrical connection path enabling activation of the phototherapy lamp, in accordance with an embodiment of the present invention.

FIG. 7 illustrates a system showing a phototherapy packaging device structure and a phototherapy device, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention will now be described in detail with reference to the accompanying drawings, wherein like reference numerals designate like or corresponding components throughout.

Embodiments of the present invention disclose a phototherapy device packaging structure designed to reduce user experience costs and enhance promotional efficiency by providing a multi-functional packaging capable of simulating the operation of the actual phototherapy device. The packaging structure comprises a packaging body, a circuit board, and a phototherapy lamp, wherein the packaging body is configured to contain and protect the phototherapy device, the circuit board is disposed within the packaging body, and the phototherapy lamp is mounted on the circuit board and electrically connected thereto. The phototherapy lamp is configured to emit light outward through a light-transmitting portion formed on the packaging body, allowing users to directly experience the therapeutic effects.

In certain embodiments of the present invention, the phototherapy device packaging structure is designed as a functional replica of the phototherapy device that it houses. The phototherapy device packaging structure not only encloses and protects the device during transportation and sale but also simulates its physical appearance, stimulation pattern, and operational behavior, thereby serving as an intelligent demonstration model for the user. This design allows the user to experience the essential features of the phototherapy device, such as light wavelength, distribution pattern, and stimulation sensation, without activating the actual therapeutic unit.

In an embodiment, the packaging body includes a first connecting portion and a second connecting portion, each having a conductive portion electrically connected to the circuit board. When the connecting portions are bent toward each other, the conductive portions contact one another, forming a closed circuit with the circuit board, the battery, and the phototherapy lamp, thereby activating the lamp. In the unbent state, the conductive portions remain separated to prevent unintended activation. The packaging body is a box body with a cover, where the cover seals a storage cavity containing the phototherapy device, and the light-transmitting portion is aligned with the phototherapy lamp. A protective film may be applied over the light-transmitting portion to prevent contamination before activation.

The packaging body is configured in flexible form factors, such as a strip, pouch, blister pack, or foldable display card. In such embodiments, the circuit board may comprise a flexible printed circuit (FPC), enabling integration into thin or deformable packaging while maintaining reliable electrical connections for the phototherapy lamp.

The phototherapy lamp comprises multiple light-emitting diodes of different wavelengths, such as red, blue, and near-infrared LEDs, to simulate various therapy modes of the device. The phototherapy lamp is configured to activate sequentially or selectively to demonstrate the distinct effects of different wavelengths, providing a richer user experience.

In an embodiment, alternative activation mechanisms are incorporated into the packaging structure. These include magnetic contacts, peel-away conductive stickers, pressure-sensitive switches, or capacitive touch sensors, which allow the phototherapy lamp and one or more stimulation elements to be activated without the need for mechanical bending of conductive portions. These mechanisms may be used independently or in combination with the first connecting portion and the second connecting portion.

In an embodiment, the packaging structure incorporates one or more stimulation elements to enhance the sensory experience. The one or more stimulation elements may include thermal modules such as thin-film heaters providing warming sensation, vibration modules using miniature motors or piezo actuators to generate tactile feedback, cooling modules using Peltier devices or gel inserts to simulate alternating hot-cold therapy, aromatherapy modules releasing fragrances from microcapsules or strips, micro-current electrodes, EMS (Electrical muscle stimulation) element, PEMF (Pulsed electromagnetic field element) and acoustic modules providing sound cues. These stimulation elements are integrated into the packaging body and are electrically connected to the circuit board, enabling synchronized or independent operation with the phototherapy lamp.

In one embodiment, the stimulation element integrated within the packaging structure is configured to replicate the functionality, operational parameters, and response behavior of the stimulation element present within the actual phototherapy device. For example, if the phototherapy device employs a microcurrent electrode, vibration motor, or thermal stimulation pad to deliver therapy, the packaging structure incorporates a corresponding stimulation element of similar configuration but lower power and simplified circuitry. This replication enables the user to perceive the same type of stimulus—such as mild vibration, warmth, or electrical pulse—that would be generated during normal therapeutic operation, thus creating a realistic demonstration experience while maintaining safety and cost efficiency.

The packaging structure includes enhanced light transmission features, such as diffusers, micro-lens arrays, or patterned transparent portions, to ensure uniform illumination, optimize light output, and create aesthetic or branding effects.

The conductive elements of the first connecting portion and the second connecting portion are formed using metal plating, printed conductive inks, or integrally formed metal components, while the circuit board is rigid or flexible, depending on the form factor of the packaging. These configurations ensure reliable electrical connectivity, reduce assembly complexity, and allow for scalable, cost-effective production.

The packaging body further includes digital interaction modules such as QR codes, NFC tags, or Bluetooth beacons integrated into the packaging body, allowing users to access tutorials, promotional content, or real-time guidance during activation.

The phototherapy device packaging structure of the present invention provides a comprehensive solution for safe, cost-effective, and interactive product demonstration. By combining light emission, optional multi-sensory stimulation, flexible activation mechanisms, and adaptable form factors, the invention allows consumers to experience the therapeutic effects of a phototherapy device directly through its packaging, thereby lowering user experience costs, improving promotional efficiency, and enabling a wide range of applications across different product types.

Several embodiments of the present invention will now be described in detail with references to FIGS.

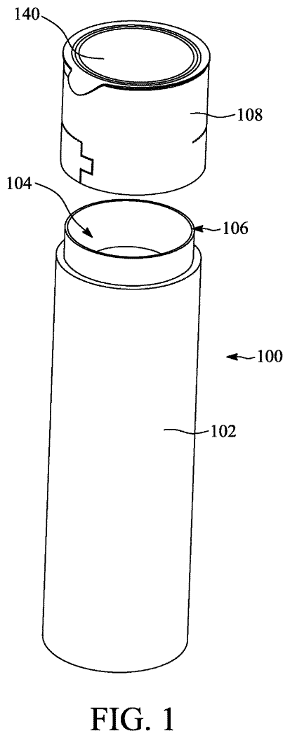

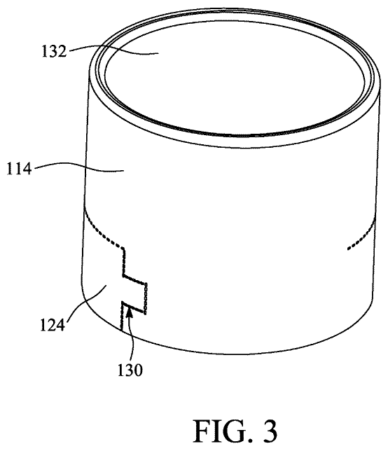

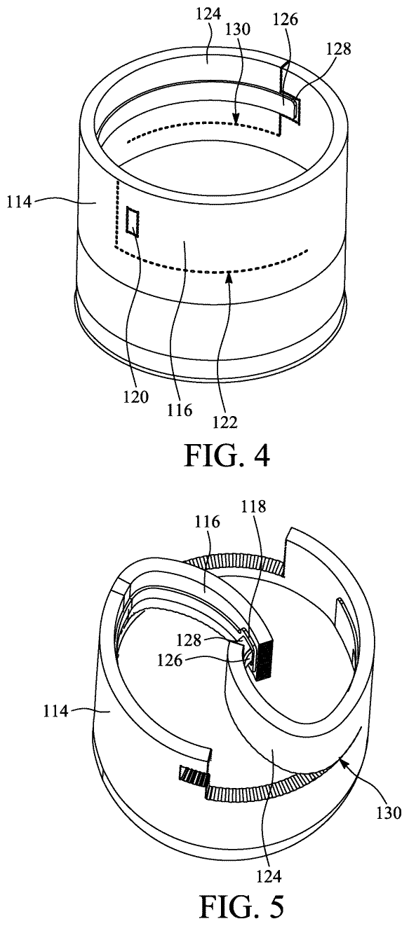

Referring to FIG. 1 to FIG. 6, the phototherapy device packaging structure comprises a packaging body 100, a circuit board 134, and a phototherapy lamp 136. The packaging body 100 is configured to house the phototherapy device, while the circuit board 134 and the phototherapy lamp 136 are disposed within the packaging body 100, the latter being electrically connected to the circuit board 134. The phototherapy lamp 136 is configured to emit light outward from the packaging body 100 through a corresponding light-transmitting portion, which is a transparent cover 132. A protective film 140 is applied over the transparent cover 132 to prevent contamination before activation.

This configuration allows users to experience the phototherapy lamp 136 directly from within the packaging body 100 during product promotion and trial use. Since the phototherapy lamp 136 is intended primarily for demonstration purposes, its operational requirements, such as lifespan or high-end functionality, are minimal. Accordingly, the structure can be simplified using basic circuitry and light-emitting components, thereby reducing both production and user experience costs.

The packaging body 100 may take the form of a rigid packaging box, such as one made of paper or plastic, or may alternatively comprise a wrapping cloth or paper. In the case of flexible packaging, the phototherapy device is enclosed within the wrapping material, which is selected to provide adequate structural integrity. The phototherapy lamp 136 may be positioned either within the packaging body 100, with a light-transmitting structure allowing passage of light, or externally on the packaging surface, in which case a light-transmitting portion is unnecessary.

In some embodiments, the packaging structure further includes a battery 138 electrically connected to the circuit board 134, enabling the phototherapy lamp 136 to operate independently without an external power source. The circuit board 134 may be implemented as a flexible circuit board to facilitate installation, reduce cost, and accommodate deformations of the packaging body 100.

In an embodiment, the packaging structure may incorporate an electrical connector, such as a female socket or exposed electrode pads, electrically connected to the circuit board 134 and accessible from the exterior of the packaging body 100. This allows the phototherapy lamp 136 to be powered from an external source, providing flexibility and simplifying manufacturing.

In some embodiments, the packaging body 100 includes a first connecting portion 116 and a second connecting portion 124. The first connecting portion 116 carries a first conductive portion 118, while the second connecting portion 124 carries a second conductive portion 126, both electrically connected to the circuit board 134 and exposed externally. The first connecting portion 116 and the second connecting portion 124 are configured to bend toward each other such that the first conductive portion 118 and the second conductive portion 126 come into contact, forming a closed circuit connecting the first conductive portion 118, the second conductive portion 126, the circuit board 134, the battery 138, and the phototherapy lamp 136.

In normal packaging conditions, the first connecting portion 116 and the second connecting portion 124 are separated, preventing the first conductive portion 118 and the second conductive portion 126 from contacting each other and thus keeping the circuit open. To activate the phototherapy lamp 136 for user experience, the first connecting portion 116 and the second connecting portion 124 are brought together, establishing contact between the first conductive portion 118 and the second conductive portion 126 and forming a closed circuit. This design eliminates the need for a separate switch, simplifies the circuitry, and ensures controlled activation of the phototherapy lamp 136. This design eliminates the need for a separate switch on the packaging body 100, thereby simplifying the circuit structure and reducing production costs.

In certain embodiments, the packaging body 100 is a box provided with a first tear line 122 and a second tear line 130, which, when torn, form the first connecting portion 116 and the second connecting portion 124, respectively. During use, the first connecting portion 116 is separated from the packaging body 100 along the first tear line 122, and the second connecting portion 124 is separated along the second tear line 130. The separated first connecting portion 116 and the second connecting portion 124 can then be brought closer together, allowing the first conductive portion 118 and the second conductive portion 126 to contact each other. This arrangement maintains the relative positions of the first connecting portion 116 and the second connecting portion 124 during normal packaging, preventing accidental contact of the first conductive portion 118 and the second conductive portion 126 and inadvertent circuit closure.

In alternative embodiments, only one of the first tear line 122 or the second tear line 130 may be provided, forming a single connecting portion (the first connecting portion 116 or the second connecting portion 124), while the other portion remains integral with the packaging body 100. The first tear line 122 and the second tear line 130 may be implemented as a series of spaced small holes resembling dashed lines, as creases, or as recesses in the packaging material, with reduced thickness at the tear line to facilitate separation.

In certain embodiments, the first connecting portion 116 includes an insertion hole 120, while the second connecting portion 124 includes an insertion tongue 128, which engages with the insertion hole 120 when bent. This configuration ensures a stable mechanical and electrical connection between the first connecting portion 116 and the second connecting portion 124, reducing the likelihood of power interruption during use. Alternatively, hooks or other mechanical connectors may be employed to secure the first connecting portion 116 and the second connecting portion 124.

In some embodiments, the first conductive portion 118 is annular, surrounding the insertion hole 120, and the second conductive portion 126 is a conductive sheet located on one side of the insertion tongue 128. When the insertion tongue 128 is inserted into the insertion hole 120, the conductive sheet establishes reliable electrical contact with the first conductive portion 118, minimizing the risk of circuit interruption and enhancing user experience.

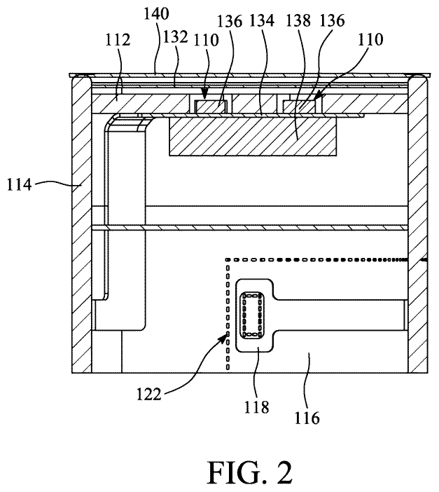

Referring to FIG. 2, a cross-sectional view of the cover portion of the packaging structure is shown. The cover has a top wall 112 and a peripheral wall 114 to define a storage cavity for housing the phototherapy lamp 136. The top wall has a light-transmitting portion 110 formed on its outer side. A protective film 140 is disposed on the outer surface of the light-transmitting portion 110 to safeguard the underlying structure prior to use.

A circuit board 134 is positioned within the cover, which is connected to the phototherapy lamp 136; the phototherapy lamp is configured to emit light outward through the light-transmitting portion 110. The phototherapy lamp 136 may include one or more LEDs or similar light-emitting elements. The top wall 112 of the cover is provided with a transparent cover 132, which is positioned over the light-transmitting portion 110. A protective film 140 is applied over the transparent cover 132 to prevent contamination before activation.

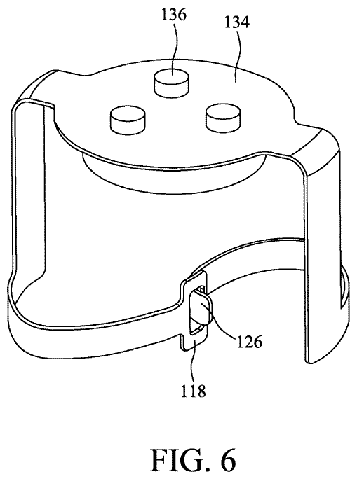

As illustrated in FIG. 6, the base plate of the circuit board 134 may be integrally formed with the first conductive portion 118 and the second conductive portion 126 as a single metal component, with the circuit board 134 concealed within the packaging body 100. This ensures structural integrity and improves the reliability of the first conductive portion 118 and the second conductive portion 126. In this embodiment, the base plate connects the conductive portions via two metal strips. In alternative embodiments, the first conductive portion 118 and the second conductive portion 126 may be formed as separate metal components electrically connected to the circuit board 134 via wires. This configuration allows independent positioning of the first conductive portion 118 and the second conductive portion 126 relative to the circuit board 134, reducing assembly constraints and facilitating flexible integration within the packaging structure.

In alternative embodiments, the first conductive portion 118 and the second conductive portion 126 may be metal-plated to enhance conductivity and reliability. In certain embodiments, the packaging body 100 comprises a box body 102 and a cover 108. The box body 102 defines a storage cavity 104 and a storage opening 106 in communication with the storage cavity 104. The cover 108 is configured to close the storage opening 106. A light-transmitting portion 110 is formed on the side of the cover 108 opposite the storage opening 106, with a circuit board 134 disposed within the cover 108 and facing the light-transmitting portion 110. The phototherapy lamp 136 is mounted on the circuit board 134 and aligned with the light-transmitting portion 110.

In certain embodiments, the cover 108 may house one or more stimulation elements, such as miniature heaters, vibration modules, cooling inserts, aromatherapy strips, or acoustic modules, integrated with the circuit board 134. These elements can be activated simultaneously or independently with the phototherapy lamp 136 to enhance the user experience. The placement of such stimulation elements within the cover 108 allows the packaging to serve as an interactive demonstration device, providing sensory feedback without requiring removal of the phototherapy device from the packaging.

During packaging, the phototherapy device is placed within the storage cavity 104, and the storage opening 106 is closed by the cover 108. For promotional or trial purposes, the cover 108 may be removed, allowing the phototherapy lamp 136 mounted on the cover 108 to be used for user experience. Since the phototherapy lamp 136 is primarily intended for demonstration, its structure can be simplified using basic circuitry and light-emitting components, thereby minimizing cost. The box-and-cover configuration ensures reliable protection of the phototherapy device while providing a secure and convenient housing for the circuit board 134 and phototherapy lamp 136. Moreover, the cover 108, being smaller and simpler in structure compared to the box body 102, can be produced at a lower cost while maintaining effective functionality for user demonstration.

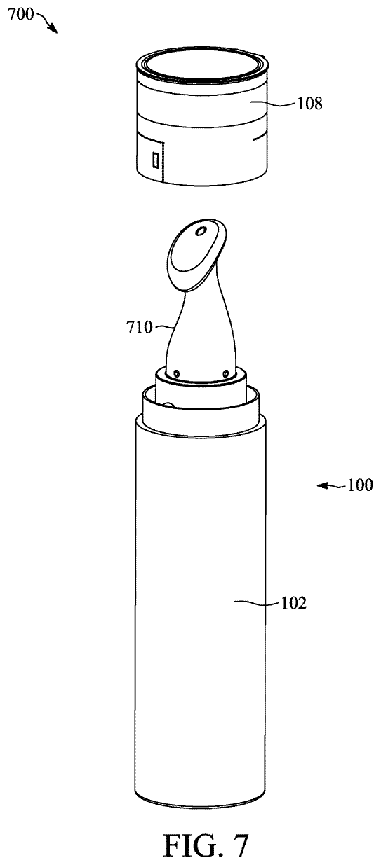

In accordance with an embodiment of the present invention, a phototherapy system 700 is provided, as shown schematically in FIG. 7. The phototherapy system 700 generally comprises a phototherapy device 710 configured to deliver therapeutic light to a user for treatment, and the packaging body 100 configured both to house the phototherapy device 710 and to provide an auxiliary demonstration or low-intensity stimulation function.

The phototherapy device 710 may be any therapeutic unit configured to emit light energy within one or more predetermined wavelength ranges suitable for photobiomodulation, wound healing, cosmetic rejuvenation, or other clinical or wellness applications. In one embodiment, the phototherapy device 710 includes an array of light-emitting diodes (LEDs), laser diodes, or hybrid optical emitters generating visible, infrared, or near-infrared light. The phototherapy device 710 may include an internal control module, a power supply, and a communication interface for interaction with the packaging body 100.

The packaging body 100 is in form of shape, such as a box, sleeve, or casing formed of paper, polymer, or composite material. The circuit board 134 is disposed within the packaging body 100 and carries a phototherapy lamp 136 electrically connected thereto. The phototherapy lamp 136 is oriented toward a light-transmitting portion 110 of the packaging body 100 or its cover 108 so that, when activated, light is emitted outwardly from the packaging body. In this configuration, the packaging body 100 operates as an interactive enclosure that visually reproduces or simulates the illumination characteristics of the phototherapy device 710.

In one embodiment, the phototherapy lamp 136 of the packaging body 100 emits light of substantially the same wavelength characteristics as the phototherapy device 710, thereby enabling a user to perceive the therapeutic color or radiance representative of the actual device output.

In another embodiment, the phototherapy lamp 136 is arranged as an array of emitters having a spatial layout corresponding to that of the emitters on the phototherapy device 710, such that the emitted light field closely replicates the optical distribution of the treatment head. This arrangement allows the packaging body 100 to serve as a realistic demonstration platform for marketing or training purposes.

The battery 138 or other portable power source may be integrated within the packaging body 100 and electrically connected to the circuit board 134 to supply energy to the phototherapy lamp 136 and any auxiliary components. Alternatively, an external electrical connector may be provided on the packaging body 100 for coupling with an external adapter, USB port, or inductive charging pad.

In certain embodiments, the circuit board 134 is implemented as a flexible printed circuit, enabling it to conform to the shape of the packaging body 100 and to accommodate bending or folding operations without damage.

The packaging body 100 may further comprise a first connecting portion 116 and a second connecting portion 124, each provided with a conductive contact. The first and second connection portions are formed integrally with the packaging body 100 and configured to move from a separated state to a contacting state. When the packaging body 100 is folded or pressed, the conductive contacts engage one another to complete an electrical circuit between the circuit board 134, the battery 138, and the phototherapy lamp 136. In this way, the light source is automatically activated by mechanical interaction with the packaging, eliminating the need for a discrete switch and thereby simplifying assembly and reducing manufacturing cost.

In certain embodiments, the packaging body 100 further includes one or more stimulation elements mounted adjacent to or integrated with the phototherapy lamp 136. The stimulation elements may include microcurrent electrodes, piezoelectric vibrators, or thermoelectric heaters configured to deliver gentle tactile, electrical, or thermal feedback when activated. This optional configuration enhances the experiential realism of the phototherapy demonstration and may also provide mild therapeutic benefit when used in proximity to the skin.

In further embodiments, one or more skin-parameter sensors are incorporated within or on the surface of the packaging body 100. The sensors may be optical reflectance sensors, temperature sensors, or bio-impedance detectors configured to measure physiological parameters of the user's skin. The sensor data may be transmitted wirelessly or via wired connection to the phototherapy device 710 or to an external computing unit for adaptive treatment control, calibration, or data analytics.

During use, the phototherapy device 710 is placed within the storage cavity of the packaging body 100. When the packaging body 100 is pressed or folded such that the first connecting portion 116 and the second connecting portion 124 contact, the circuit closes and activates the phototherapy lamp 136 and, if present, the stimulation element. Light is emitted outwardly through the light-transmitting portion 110, permitting a user to view or experience the therapeutic illumination effect directly from the packaging. The system thus enables a dual-function operation, serving as both protective packaging and a demonstration apparatus that authentically represents the performance of the phototherapy device 710.

In certain alternative embodiments, the present invention provides an enhanced phototherapy system incorporating a smart packaging structure designed to function not only as a physical container but also as an intelligent demonstration platform. The smart packaging structure may include additional electronic components configured to perform monitoring, sensing, and communication functions for improved user engagement and demonstration fidelity.

In one embodiment, both the phototherapy device and the packaging structure are configured to emit light having substantially identical wavelength characteristics, such that the packaging system accurately represents the therapeutic light output of the actual device. This correspondence enables a realistic visual and perceptual demonstration without requiring the full treatment-level power output of the device. The wavelength match may be achieved by using the same class of light-emitting diodes or equivalent photonic components in both the device and the packaging structure.

In an embodiment, the stimulation element integrated within the phototherapy device packaging structure is configured to replicate the functionality, operational parameters, and response behavior of the stimulation element present within the actual phototherapy device. For example, if the phototherapy device employs a microcurrent electrode, vibration motor, or thermal stimulation pad to deliver therapy, the packaging structure incorporates a corresponding stimulation element of similar configuration but lower power and simplified circuitry. This replication enables the user to perceive the same type of stimulus, such as mild vibration, warmth, or electrical pulse that would be generated during normal therapeutic operation thus creating a realistic demonstration experience while maintaining safety and cost efficiency.

Furthermore, the spatial arrangement of the light-emitting and stimulation elements on the phototherapy device packaging structure mirrors that of the phototherapy device. In one exemplary configuration, if the phototherapy device includes a central stimulation element surrounded by a plurality of LED beads arranged in a circular or polygonal pattern, the packaging structure reproduces this pattern in a miniaturized layout. The central portion of the packaging corresponds to the stimulation element, while the surrounding region contains multiple LED beads disposed in a geometrically similar manner. This one-to-one correspondence between the element arrangement of the phototherapy device and that of its packaging structure provides a visual and functional replica, allowing the user to directly associate the demonstration light pattern with the real treatment configuration.

In another embodiment of the present invention, the phototherapy device packaging structure mimics the illumination configuration of the phototherapy device. For instance, the phototherapy device comprises a plurality of first light-emitting diodes (LEDs) configured to emit red light within a wavelength range of approximately 660-680 nanometers (nm), and a plurality of second LEDs configured to emit infrared (IR) light within a wavelength range of approximately 1000-1050 nm. To ensure consistency between the phototherapy device and its phototherapy device packaging structure, the phototherapy device packaging structure is correspondingly configured to mimic the dual-wavelength illumination pattern of the housed device. Specifically, the phototherapy device packaging structure incorporates first light sources that emit light in the red wavelength range (around 670 nm) and second light sources that emit in the infrared range (around 1020 nm). These light sources are spatially arranged on the packaging structure in a pattern identical to or proportionally scaled from that of the phototherapy device. For instance, if the phototherapy device arranges red LEDs and infrared LEDs alternately in a circular or grid pattern around a central stimulation element, the phototherapy device packaging structure reproduces the same arrangement in a miniaturized or simplified configuration.

The relative ratio and spatial distribution of the red and infrared LEDs in the phototherapy device packaging structure are designed to replicate the optical emission balance of the phototherapy device. The intensity levels may be adjusted to lower demonstration-safe values while maintaining the same spectral characteristics, thereby allowing the user to visually perceive the red illumination and indirectly experience the warmth associated with infrared emission. Although the infrared light of the phototherapy device packaging structure may be of reduced power for safety and efficiency reasons, the emission wavelength is preserved to authentically simulate the functional behavior of the phototherapy device.

In another embodiment, the outer contour and form factor of the packaging body are designed to resemble the shape and ergonomics of the phototherapy device it contains. For instance, if the phototherapy device has a curved, mask-like, handheld, or panel-type body, the packaging structure adopts a corresponding external geometry. This not only enhances brand consistency and product recognition but also provides the user with an authentic tactile interaction. The replication of external form ensures that the demonstration device reflects the proportions, curvature, and orientation of the actual product, thereby reinforcing the immersive experience.

The phototherapy system may include a removable or external battery module configured to power the phototherapy lamp within the packaging structure. The removable battery allows the packaging to remain functional even during prolonged retail display or repeated demonstration use. In certain versions, the battery module may be rechargeable or interchangeable with that of the phototherapy device, thereby reducing maintenance and ensuring energy efficiency.

In another embodiment, the packaging structure may be equipped with one or more sensors configured to detect parameters of the user's skin, such as temperature, reflectivity, impedance, or moisture content. The detected parameters may be used to provide real-time or simulated feedback for adaptive control of the phototherapy lamp, thereby mimicking personalized treatment conditions. The sensor data may also be used to activate or adjust the light intensity or duration of the demonstration based on user proximity or interaction.

In certain versions, the packaging structure may act as a “Smart Demo Device”, capable of recording, analyzing, and transmitting user interaction data. A data processor disposed within the packaging structure may determine one or more user engagement parameters, such as the number of activations of the demonstration light, duration of interaction, user proximity, or user feedback rating. This data can be stored locally or transmitted for further analysis.

The smart demo device may further include a wireless communication module, such as a Bluetooth® or Wi-Fi transceiver, or an NFC tag configured to communicate with a mobile phone, the phototherapy device, or a remote server. Through such communication, the system may transmit engagement parameters to a manufacturer or user application, enabling remote monitoring, marketing analytics, or firmware synchronization. In one implementation, scanning the NFC tag using a mobile device may automatically open a web page, download an application, or trigger a demo mode on the phototherapy device itself.

The processor may further analyze engagement data to determine behavioral trends, such as the average number of activations or the typical duration of a user's interaction with the packaging. Based on these metrics, the system may autonomously adjust its demonstration parameters, for example by modifying the brightness, wavelength pattern, or emission duration to enhance user experience or conserve power. The ability to adaptively modify operational parameters based on engagement data improves product presentation and marketing effectiveness.

In another embodiment, the smart packaging may include a feedback interface, such as a capacitive touch surface, button input, or voice-responsive module, allowing the user to provide simple feedback signals (e.g., satisfaction rating or interest level). These inputs may be recorded by the processor and transmitted via the wireless communication module to a central server or user device for further analysis. The integration of feedback with engagement data allows manufacturers to refine treatment algorithms or optimize packaging design based on real user interactions.

Collectively, these structural and functional replications allow the packaging structure to operate as a scaled, interactive, and cost-efficient simulation model of the actual phototherapy device. It delivers both visual and sensory cues that accurately represent the therapeutic output and user experience, while minimizing power consumption, circuitry complexity, and manufacturing costs. This design also supports the marketing and educational objectives of the manufacturer by enabling potential users to understand and experience the phototherapy principles without engaging the main therapeutic hardware.

Through these additional embodiments, the packaging structure transcends its traditional protective function and operates as an interactive, data-enabled, and adaptive demonstration system. By integrating sensors, processors, removable power modules, and wireless communication elements, the phototherapy system achieves improved user engagement, more accurate representation of treatment conditions, and greater utility in promotional, clinical, and research applications.

In accordance with another embodiment of the present invention, a method of operating a phototherapy system 700 is provided. The method enables a user or manufacturer to utilize the packaging body 100 as an interactive demonstration platform for the phototherapy device 710 while maintaining the functional integrity of the device during storage, transport, or retail display.

The method includes the step of placing the phototherapy device 710 within the packaging body 100, such that the device is received within a storage cavity of the packaging body 100. The packaging body 100 may include cushioning or guiding features to ensure proper orientation of the phototherapy device 710 relative to the light-transmitting portion 110 and the circuit board 134. This arrangement allows the packaging to function as both a protective enclosure and a demonstration interface.

The method further includes activating a phototherapy lamp 136 mounted within the packaging body 100, wherein the phototherapy lamp 136 is electrically connected to the circuit board 134 and powered either by an internal battery 138 or by an external power connector. Activation may occur automatically upon user interaction with the packaging structure, for example, by folding, pressing, or deforming the packaging body 100 so as to bring the first connecting portion 116 and second connecting portion 124 into contact, thereby completing a closed electrical circuit. In other embodiments, activation may be controlled electronically or wirelessly via a communication interface with the phototherapy device 710.

Once activated, the phototherapy lamp 136 emits light outwardly from the packaging body 100, typically through the light-transmitting portion 110. The emitted light preferably possesses wavelength characteristics substantially identical to those of the phototherapy device 710, thereby providing a realistic optical representation of the device's therapeutic emission. In some embodiments, the emitted wavelength may be in the visible, infrared, or near-infrared range depending on the target treatment application.

The method may optionally include allowing a user to expose a region of the skin to the emitted light from the phototherapy lamp 136 to experience the warmth, brightness, or mild photo biological effect associated with the actual therapy device. Because the lamp within the packaging body 100 is designed for demonstration, it typically operates at reduced intensity and limited duration, ensuring safety for casual use.

In another aspect, the method includes recording user-interaction data associated with the demonstration, such as the number of activations, duration of exposure, proximity detection, or user touch data. The recorded data may be stored in a memory module on the circuit board 134 or transmitted wirelessly to a remote server, manufacturer platform, or mobile application. Such data collection may be useful for marketing analytics, consumer engagement metrics, or remote diagnostics.

In some embodiments, the method further includes measuring skin parameters through one or more sensors embedded in or on the surface of the packaging body 100. The sensors may collect information such as surface temperature, reflectivity, or impedance, which can be transmitted to the phototherapy device 710 or to an associated control system. The phototherapy device 710 may use these data inputs to adjust light intensity, duration, or wavelength dynamically for adaptive control.

In a further embodiment, the method may comprise activating additional stimulation elements disposed in the packaging body 100. The stimulation elements may include microcurrent electrodes, vibration modules, or thermal emitters that operate concurrently with or independently from the phototherapy lamp 136. These elements provide tactile or sensory cues that simulate the user experience of actual treatment, thereby enhancing engagement during product demonstration.

Following the demonstration, the packaging body 100 may automatically deactivate after a preset duration or upon separation of the first connection portion and second connection portion, thereby interrupting the closed circuit and conserving power. The phototherapy device 710 remains securely enclosed within the packaging body 100 and can subsequently be removed for normal therapeutic use.

Through the above method, the packaging body 100 not only protects and presents the phototherapy device 710 but also operates as a functional simulation platform capable of demonstrating therapeutic light emission, collecting user data, and optionally providing low-level stimulation or sensor feedback. The method thereby enhances user interaction, improves product education, and offers manufacturers a valuable tool for experiential marketing and feedback-based product refinement.

The phototherapy device packaging structure of the present invention is primarily designed for promotional, demonstration, and user-experience applications. By incorporating a phototherapy lamp within the packaging, potential customers can directly experience the therapeutic effect of the device without the need to unbox or fully operate the phototherapy product. This enables manufacturers and retailers to reduce product trial costs, minimize wastage, and provide a controlled, interactive demonstration that highlights the device's benefits effectively. The inclusion of additional stimulation elements, such as miniature heaters, vibration modules, or aromatherapy inserts, further enhances the promotional impact by offering a multisensory user experience, simulating the actual device operation in a compact, cost-effective format.

In addition to marketing and promotional applications, the invention can be applied in retail packaging for consumer convenience, allowing users to engage with the phototherapy product immediately upon purchase. The modular design, including features such as tear lines, connecting portions, and light-transmitting structures, provides flexibility and reliability in packaging while enabling optional activation of light or stimulation features. The invention thus finds utility in contexts where interactive demonstration, cost-efficient user trials, and enhanced user engagement are desirable, including in pharmacies, beauty stores, health and wellness outlets, and product launch events.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A phototherapy device packaging structure, comprising:
a packaging body configured to house a phototherapy device;
a circuit board disposed within the packaging body; and
a phototherapy lamp mounted within the packaging body and electrically connected to the circuit board, the phototherapy lamp being configured to emit light outward from the packaging body.

2. The phototherapy device packaging structure of claim 1, further comprising a battery electrically connected to the circuit board.

3. The phototherapy device packaging structure of claim 2, wherein the packaging body comprises a first connection portion and a second connection portion, each of the first and second connection portions having a conductive member electrically connected to the circuit board, and wherein the first and second connection portions are movable between a separated state and a contacting state, wherein when in the contacting state, the conductive members contact each other to form a closed electrical circuit between a power source and the phototherapy light source, thereby activating the phototherapy light source.

4. The phototherapy device packaging structure of claim 3, wherein the packaging body is a box having a first tear line and a second tear line, wherein the first connecting portion and the second connecting portion are separated from the box along the respective tear lines.

5. The phototherapy device packaging structure of claim 4, wherein the first connecting portion includes an insertion hole, and the second connecting portion includes an insertion tongue configured to engage the insertion hole when bent, thereby forming a stable mechanical and electrical connection.

6. The phototherapy device packaging structure of claim 3, wherein the conductive portions are selected from the group consisting of integrally formed metal components, metal components electrically connected via wires, and metal-plated components.

7. The phototherapy device packaging structure of claim 1, wherein the packaging body comprises a box body and a cover, the box body defining a storage cavity and a storage opening, the cover configured to close the storage opening, the cover including a light-transmitting portion, and the phototherapy lamp is aligned with the light-transmitting portion.

8. The phototherapy device packaging structure of claim 7, further comprising a protective film affixed to the cover and covering the light-transmitting portion, wherein the light-transmitting portion is exposed upon removal of the protective film.

9. The phototherapy device packaging structure of claim 7, further comprising one or more stimulation elements mounted within the cover and electrically or mechanically coupled to the circuit board, the stimulation elements being selected from the group consisting of miniature heaters, vibration modules, cooling inserts, aromatherapy inserts, micro-current electrodes, EMS (Electrical Muscle Stimulation) element, PEMF (Pulsed electromagnetic field) element, and acoustic modules.

10. The phototherapy device packaging structure of claim 1, further comprising an electrical connector electrically connected to the circuit board and exposed outside the packaging body, the connector configured to supply external power to the phototherapy lamp.

11. The phototherapy device packaging structure of claim 1, wherein the phototherapy lamp comprises multiple LEDs configured to emit light at different wavelengths.

12. A phototherapy system, comprising:
a phototherapy device configured to emit therapeutic light toward a user for treatment; and
a packaging structure for the phototherapy device, the packaging structure comprising:
a packaging body;
a circuit board disposed within the packaging body; and
a phototherapy lamp mounted within the packaging body and electrically connected to the circuit board, the phototherapy lamp is configured to emit light outwardly from the packaging body.

13. The phototherapy system of claim 12, further comprising a battery electrically connected to the circuit board and configured to supply electrical power to the phototherapy lamp.

14. The phototherapy system of claim 12, wherein the phototherapy lamp of the packaging structure comprises one or more light-emitting diodes configured to emit light having wavelength characteristics substantially identical to those of the phototherapy device,
whereby the emitted light replicates the treatment wavelength of the phototherapy device for demonstration or user experience.

15. The phototherapy system of claim 14, wherein the phototherapy lamp comprises an array of light-emitting diodes spatially arranged in a pattern corresponding to an array of therapeutic emitters of the phototherapy device,
whereby the emitted light field and spatial distribution pattern of the packaging structure simulate those of the phototherapy device to provide a realistic phototherapy demonstration.

16. The phototherapy system of claim 12, wherein the packaging structure includes a first connection portion and a second connection portion, each comprising a conductive contact,
the first and second connection portions being integrally formed with the packaging body and movable between a separated state and a contacting state,
such that folding, bending, or pressing the packaging structure causes the conductive contacts to engage and complete a closed circuit to activate the phototherapy lamp.

17. The phototherapy system of claim 12, further comprising at least one skin-parameter sensor disposed within the packaging structure and configured to measure a skin parameter of the user,
wherein the packaging structure is configured to transmit the measured parameter to the phototherapy device for adaptive treatment control.

18. A method of operating a phototherapy system, the system comprising a phototherapy device and a packaging structure including a packaging body, a circuit board, and a phototherapy lamp mounted within the packaging body, the method comprising:
placing the phototherapy device within the packaging structure;
activating the phototherapy lamp mounted within the packaging structure to emit light outwardly from the packaging body;
emitting light from the phototherapy lamp having wavelength characteristics substantially identical to those of the phototherapy device, thereby providing a demonstration of therapeutic light emission representative of the phototherapy device; and
optionally allowing a user to expose skin to the emitted light from the packaging structure to experience the therapeutic light effect without activating the phototherapy device.

19. The method of claim 18, further comprising folding or pressing the packaging structure to bring a first connection portion and a second connection portion into electrical contact, thereby completing a closed circuit to power the phototherapy lamp.

20. The method of claim 18, further comprising recording user-interaction data associated with the demonstration, the data including at least one of a number of activations, duration of light emission, or user proximity,
and transmitting the recorded data to a remote server or manufacturer system for analytics or marketing purposes.
